(19) United States
(12) Patent Application Publication
(10) Pub. No.: US 2020/0368531 A1
(43) Pub. Date: Nov. 26, 2020

(54) TREATMENT OF EYE DISORDERS

(71) Applicants: GALVANI BIOELECTRONICS LIMITED, Brentford, Middlesex (GB); UNIVERSITY OF SOUTHERN CALIFORNIA, Los Angeles, CA (US)

(72) Inventors: Arun SRIDHAR, Stevenage (GB); Andrew C WEITZ, Los Angeles, CA (US); Victor Eugene PIKOV, Stevenage (GB); Alessandra GIAROLA, Stevenage (GB); Mark HUMAYUN, Los Angeles, CA (US)

(73) Assignees: Galvani Bioelectronics Limited, Brentford, Middlesex (GB); University of Southern California, Los Angeles, CA (US)

(21) Appl. No.: 16/634,252

(22) PCT Filed: Jul. 27, 2018
(86) PCT No.: PCT/US2018/044190

§ 371 Date: Jan. 27, 2020

Related U.S. Application Data
(60) Provisional application No. 62/538,493, filed on Jul. 28, 2017.

(51) Int. Cl. A61N 1/36; A61N 1/05

(52) U.S. Cl. CPC A61N 1/36135; A61N 1/0526; A61N 1/36171; A61N 1/3615; A61N 1/3606

(57) ABSTRACT

Modulation of neural signaling of an eye-related sympathetic nerve can decrease the levels of pro-inflammatory cytokines in the eye, and this provides a way of treating eye disorders, such as ocular neovascular diseases.

### CROSS REFERENCE TO RELATED APPLICATIONS

This application claims the benefit of U.S. Provisional Patent Application No. 62/538,493, filed Jul. 28, 2017.

### TECHNICAL FIELD

This disclosure relates to the treatment of eye disorders, more particularly to methods and medical devices that deliver electromodulation therapy for such purposes.

### BACKGROUND

Ocular neovascular diseases, such as diabetic retinopathy (DR), are the most common cause of moderate to severe vision loss in developed countries [reference ¹]. These diseases are typically treated with intraocular injections of drugs that target VEGF. The release of VEGF is thought to contribute to increased vascular permeability in the eye and inappropriate new vessel growth. The VEGF injections must be given every 4-6 weeks and carry a number of risks. The drugs are effective in slowing disease progression but do not prevent eventual vision loss. Campochiaro, P. A., Journal of Molecular Medicine, 2013; 91:311-321.

Several lines of evidence suggest a critical role of the sympathetic nervous system in maintaining ocular vascular homeostasis [reference ²] Evidence in animal models suggests that a decrease of the β-adrenergic function may result in reduction or exacerbation of the vascular changes, thus suggesting possible dual effects of β-adrenoreceptor (β-AR) modulation. There is also evidence suggesting that these vascular changes are associated with changes in the expression and secretion of angiogenic growth factors, such as vascular endothelial growth factor (VEGF) and pigment epithelium-derived factor (PEDF), which are regulated by the sympathetic nerves [references ^{2,3,4}]. Casini, et al., Progress in Retinal and Eye Reseach, 2014; 42:103-129.Wiley et al., Invest Ophthalmol Vis Sci, 2006; 47(1): 439-43.Steinle et al., Exp Eye Res, 2006; 83(1): 16-23.

These observations have prompted the use of β-AR blockers in therapy. For example, oral administration of the β1-/β2-AR blocker propranolol in clinical trials in preterm infants with retinopathy of prematurity (ROP) produced positive results in terms of efficacy, although safety problems were also reported. However, there are data demonstrating significant anti-apoptotic effects exerted by β-AR agonists; therefore if β-AR blockers were used to inhibit aberrant. neovascularization, there may be a burden to pay in terms of impaired neuronal viability [reference ²].

The disclosure aims to provide further and improved treatments of eye disorders, such as eye disorders that are associated with vascular remodeling, e.g, ocular neovascular diseases.

### SUMMARY

The inventors found that modulation of neural activity of an eye-related sympathetic nerve (e.g. the internal carotid nerve (ICN)) is capable of regulating vascular remodeling, so it provides a way to treat eye disorders, such as ocular neovascular diseases. In particular, the inventors found an increase in the TNFα levels in the retina as a result of denervation the ICN in nave rats. The results therefore suggest that applying a signal (e.g. an electrical signal) to the ICN to modulate (e.g. stimulate) the neural activity of the ICN could be an elective strategy for treating eye disorders, for example, an eye disorder that is associated with ocular neovascularization, such as retinal neovascularization (e.g. diabetic retinopathy (DR) or a neovascular disease caused by injury to the eye).

Thus, the disclosure provides a method of treating an eye disorder in a subject by reversibly modulating the neural activity of an eye-related sympathetic nerve. A preferred way of reversibly modulating (e.g. stimulating) the neural activity of the eye-related sympathetic nerve neural activity uses a device or system which applies a signal (e.g an electrical signal) to the eye-related sympathetic nerve.

The disclosure also provides a method of treating an eye disorder in a subject, comprising applying a signal to an. eye-related sympathetic nerve in the subject to reversibly modulate (e.g stimulate) the neural activity of the eye-related. sympathetic nerve.

The disclosure provides an implantable device or system according to the disclosure comprising at least. one neural interfacing element, such as a transducer, preferably an electrode, suitable for placement on, in, or around an eye-related sympathetic nerve, and a signal generator for generating a signal to be applied to the eye-related sympathetic nerve via the at least one neural interfacing element such that the signal reversibly modulates (e.g. stimulates) the neural activity of the eye-related sympathetic nerve to produce a change, preferably an. improvement, in one or more physiological parameters in the subject. The physiological parameters may be one or more of the group consisting of: the level of an angiogenic growth factor in the eye, neovascularization (e.g. retinal neovascularization), ocular blood flow, blood pressure, blood oxygenation, the extent of vision impairment, the level of an immune response modulator (e.g. a cytokine) in the eye, the extent of blood vessel leakage in the eye, the extent of macular edema, the presence of retinal exudates, the presence of capillary microaneurysms, the presence of hemorrhages, the extent of retinal cell death, the extent of capillary basement membrane thickening, the level of an oxidative stress marker, and the level of a peroxynitrite marker.

The disclosure also provides a method of treating an eye disorder in a subject, comprising: (i) implanting in the subject a device or system of the disclosure; (ii) positioning a neural interfacing element of the device or system in signaling contact with an eye-related sympathetic nerve in the subject; and optionally (iii) activating the device or system.

Similarly, the disclosure provides a method of reversibly modulating (e.g. stimulating) neural activity in an eye-related sympathetic nerve in a subject, comprising: (i) implanting in the subject a device or system of the disclosure; (ii) positioning a neural interfacing element in signaling contact with an eye-related sympathetic nerve in the subject; and optionally (iii) activating the device or system.

The disclosure also provides a method of implanting a device or a system of the disclosure in a subject, comprising; positioning a neural interfacing element of the device or system in signaling contact with an eye-related sympathetic nerve in the subject.

The disclosure also provides a device or a system of the disclosure, wherein the device or system is attached to an eye-related sympathetic nerve.

The disclosure also provides the use of a device or system for treating an eye disorder in a subject, by reversibly modulating (e.g. stimulating) the neural activity in an eye-related sympathetic nerve in the subject.

The disclosure also provides a charged particle for use in a method of treating an eye disorder, wherein the charged particle causes reversible depolarization of the nerve membrane of an eye-related sympathetic nerve, such that an action potential is generated de novo in the modified nerve.

The disclosure also provides a modified eye-related sympathetic nerve to which a neural interfacing element of the system or device of the disclosure is attached. The neural interfacing element is in signaling contact with the eye-related sympathetic nerve and so the eye-related sympathetic nerve can be distinguished from the eve-related sympathetic nerve in its natural state. Furthermore, the nerve is located in a subject who suffers from, or is at risk of, an eye disorder.

The disclosure also provides a modified eye-related sympathetic nerve, wherein neural activity is reversibly modulated (e.g. stimulated) by applying a signal to the eye-related sympathetic nerve.

The disclosure also provides a modified eye-related sympathetic nerve, wherein the nerve membrane is reversibly depolarized by an electric field, such that an action potential is generated de novo in the modified eye-related sympathetic nerve.

The disclosure also provides a modified eve-related sympathetic nerve hounded by a nerve membrane, comprising a distribution of potassium and sodium ions movable across the nerve membrane to alter the electrical membrane potential of the nerve so as to propagate an action potential along the nerve in a normal state; wherein at least a portion of the eye-related sympathetic nerve is subject to the application of a temporary external electrical field which modifies the concentration of potassium and sodium ions within the nerve, causing depolarization of the nerve membrane, thereby, in a disrupted state, temporarily generating an action potential de novo across that portion; wherein the nerve returns to its normal state once the external electrical field is removed.

The disclosure also provides a modified eye-related sympathetic nerve obtainable by reversibly modulating (e.g. stimulating) neural activity of the eye-related sympathetic nerve according to a method of the disclosure.

The disclosure also provides a method of modifying an eye-related sympathetic nerve's activity, comprising a step of applying a signal to the eye-related sympathetic nerve in order to reversibly modulate (e.g. stimulate) the neural activity of the eye-related sympathetic nerve in a subject, Preferably the method does not involve a method for treatment of the human or animal body by surgery. The subject already carries a device or system of the disclosure which is in signaling contact with the eye-related sympathetic nerve.

The disclosure also provides a method of controlling a device or system of the disclosure which is in signaling contact with the eye-related sympathetic nerve, comprising a step of sending control instructions to the device or system, in response to which the device or system applies a signal to the eye-related sympathetic nerve.

The disclosure also provides a computer system implemented method, wherein the method comprises applying a signal to an eye-related sympathetic nerve via at least one neural interfacing element, preferably an electrode, such that the signal reversibly modulates the neural activity of the eye-related sympathetic nerve to produce a change in a physiological parameter in the subject, wherein the at least one neural interfacing element is suitable for placement on, in, or around an eye-related sympathetic nerve, wherein the physiological parameter is one or more of the group consisting of; the level of an angiogenic growth factor in the eye, neovascularization (e.g. retinal neovascularization), ocular blood flow, blood pressure, blood oxygenation, the extent of vision impairment, the level of an immune response modulator (e.g. a cytokine) in the eye, the extent of blood vessel leakage in the eye, the extent of macular edema, the presence of retinal exudates, the presence of capillary microaneurysms, the presence of hemorrhages, the extent of retinal cell death, the Went of capillary basement membrane thickening, the level of an oxidative stress marker, and the level of a peroxynitrite marker.

### BRIEF DESCRIPTION OF THE DRAWINGS

FIG. 1 is a diagram of the sympathetic and parasympathetic innervation of the eye and lacrimal glands, adapted from [reference ⁵]. Sympathetic fibers (S) arise from the superior cervical ganglion (SCG) and travel along the internal carotid artery (IC), then Drummond et al., Brain, 1992; 11(5): 1429-1445. (shown as a dotted line) project to the frontal arteries (FA) and sweat glands (SG). Parasympathetic fibers (PS), originating in the superior salivatory nucleus (SSN), traverse the facial nerve (CrN7) and the greater superficial petrosal nerve (GSP) to join the vidian nerve (VN) and synapse in the sphenopalatine ganglion (SPG); postganglionic fibers then loop back as orbital rami (OR) to the cavernous sinus and internal carotid artery Where they form a retro-orbital plexus with sympathetic and trigeminal fibers, before advancing to supply the lacrimal glands (LG) and cutaneous circulation of the forehead. Also shown is the external carotid artery (EC) and the first division of the trigeminal nerve (VI).

FIG. 2 shows photographs of ICN transection in a rat. FIG. 2A shows a photograph of the surgical procedure showing transection of the left ICN. FIG. 2B shows ptosis of the ipsilateral eyelid observed 24 hours after surgery.

FIG. 3 shows TNF-α protein levels in the retina 6 weeks after unilateral ICN transection, as measured by ELISA (n=5 rats). Error bars represent SD.

FIG. 4 is a block diagram illustrating elements of a system .for performing electrical modulation in an eye-related sympathetic nerve (e.g, the ICN) according to the present disclosure.

### DETAILED DESCRIPTION OF ILLUSTRATIVE EMBODIMENTS

### An Eye-Related Sympathetic Nerve

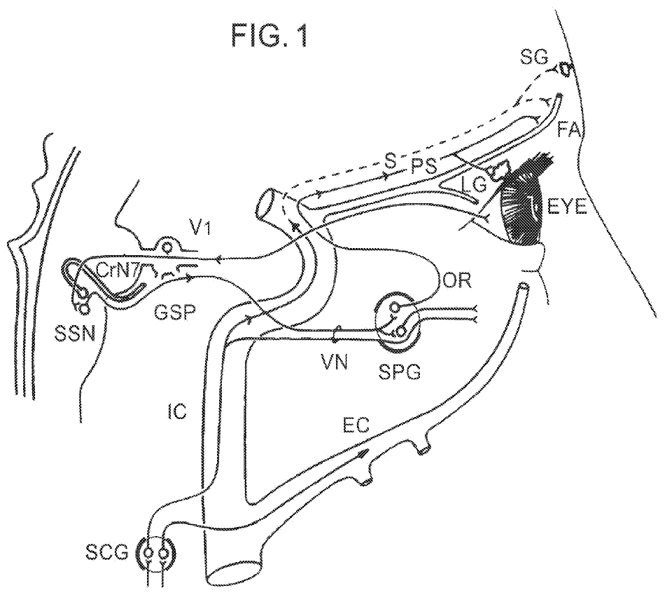

The autonomic nervous system influences numerous ocular functions [reference ⁶]including pupil diameter and ocular accommodation, ocular blood flow, and intra-ocular pressure. Sympathetic innervation of the eye arises from preganglionic neurons located in the C8-T2 segments of the spinal cord, a region termed the ciliospinal center of Budge (and Waller). The axons of these preganglionic neurons project to the sympathetic chain ganglia and travel in the sympathetic trunk to the superior cervical ganglion where they contact post ganglionic neurons. The majority of the postganglionic axons leave the superior cervical ganglion through either the external carotid nerve or the internal carotid nerve (ICN). The ICN travels along the internal carotid artery, then projects to the frontal arteries and sweat McDougal and Gamlin, 2015; Compr Physiol.; 5(1): 439-473. glands. The ICN is the eye's only source of sympathetic innervation [reference⁷, see FIG. 1].

The superior cervical ganglion lies on the transverse processes of the second and third cervical vertebrae and is possibly formed from four fused ganglia. The internal carotid artery within the carotid sheath is anterior, and longus capitis muscle is posterior. The lower end of the ganglion is united by a connecting trunk to the middle cervical ganglion. The upper end connects with the ICN [reference ⁸].

Postganglionic branches of the superior cervical ganglion are distributed in the ICN, which ascends with the internal carotid artery into the carotid canal to enter the cranial cavity, and in lateral, medial and anterior branches [references ^{8,9}].

The superior cervical ganglion is a consistent structure; human cadaveric studies show that it can be detected in every specimen on both sides [references ^{9,10,11,12,13}]. One study shows that the common carotid artery bifurcation is a good landmark for localizing the superior cervical ganglion for anaesthetic block [reference ¹⁰]. The data show that the average distance from the inferior pole of the superior cervical ganglion to the common carotid artery bifurcation is 4.1 mm (female) and 2.9 mm (male).

Parasympathetic fibers, originating in the superior salivatory nucleus, traverse the facial nerve (CrN7) and the greater superficial petrosal nerve to join the vidian nerve and synapse in the sphenopalatine ganglion; postganglionic fibers then loop back as orbital rami to the cavernous sinus and internal carotid artery where they form a retro-orbital plexus with sympathetic and trigeminal fibers, before advancing to supply the lacrimal glands and cutaneous circulation of the forehead. Smith et al., Journal of Comparative Neurology, 1990; 301:490-500,Gray's Anatomy, 41 ed.Mitsuoka, K., T. Kikutani, and I. Sato, Morphological relationship between the superior cervical ganglion and cervical nerves m Japanese endaver donors. Brain Behav, 2017. 7(2): p. e00619.Wisco, J. J., et al., A heat map of superior cervical ganglion location relative to the common carotid artery bifurcation. Anesth Analg, 2012. 14(2): p. 462-5.Fazliogullari, Z., et at. A morphometric analysis of superior cervical ganglion and its surrounding structures. Surg Radial Anat, 2016. 38(3): p. 299-302.Yin, Z., et al., Neuroanatomy and clinical analysis of the cervical sympathetic trunk and longus colli. J Biomed Res, 2015. 29(6): p. 501-7.Saylam, C. Y., et al., Neuroanatamy of cervical sympathetic trunk: a cadaveric study. Clin Anat, 2009. 22(3): p. 324-30.

Parasympathetic innervation of the eye also originates from neurons in the Edinger-Westphal preganglionic (EWpg) cell group, the autonomic subdivision of the third cranial nerve nucleus, which lies in the rostral mesencephalon. The neurons in EWpg project by way of the oculomotor (III) nerve to postganglionic cells in the ciliary ganglion.

Targets of sympathetic innervation of the eye include blood vessels (e.g. choroidal blood vessels, iris blood vessels, ciliary body blood vessels, episcleral blood vessels). The neural activity of an eye-related sympathetic nerve is naturally associated with the regulation of vascular remodeling in the eye, e.g. altering structure and arrangement in blood vessels through cell growth, cell death, cell migration and/or production or degradation of the extracellular matrix. A potential mechanism for the vascular remodeling may be alterations in the regulation of angiogenic growth factors, e.g. VEGF and PEDF.

Thus, by modulating neural activity in an eye-related sympathetic nerve, it is possible to decrease the level of pro-inflammatory cytokines in the eye, thereby assisting in treating eye conditions, such as ocular neovascular diseases. For example, stimulation of the neural activity of an eye-related sympathetic nerve can cause a decrease in the level of a pro-inflammatory cytokine (e.g. TNF-α) in the retina, and this could be an effective strategy for treating diabetic retinopathy (DR).

The disclosure can modulate activity at any site along an eye-related sympathetic nerve. For example, the site may be at the cervical portion of the sympathetic trunk, e.g. at the superior cervical ganglion. The site may be at a postganglionic sympathetic nerve projecting from the superior cervical ganglion toward the eye, such as the ICN. Alternatively, the site may be at a preganglionic eye-related sympathetic nerve in the cervical sympathetic trunk.

Preferably, the eye-related sympathetic nerve is modulated at the ICN. The disclosure may modulate at any site along the ICN. For example, the site is in the neck, and e.g. the signal is applied at the ICN in the neck. For example, the site is beneath and/or adjacent to the hypoglossal nerve in the neck. Preferably, the site is amenable for electrodes attachment.

The eye-related sympathetic nerve may be modulated at the superior cervical ganglion. Neuronal subpopulations exist in specific regions of the superior cervical ganglion. For example, the cell bodies of neurons whose axons project out the ICN are located primarily in the rostral part of the superior cervical ganglion [references ^{14,15}]. The disclosure preferably modulates these cell bodies. The disclosure preferably modulates the rostral part of the superior cervical ganglion. Li and Hom (2006) J. Neurophysiol 95: 187-195.Bowers and Zigmond (1979) 185: 381-192.

Thus, the disclosure may involve applying a signal to an eye-related sympathetic nerve, e.g. the superior cervical ganglion or the cervical portion of the sympathetic trunk, such that all the nerve fibers within the nerve are modulated. Alternatively, the disclosure may involve applying a signal to an eye-related sympathetic nerve, e.g superior cervical ganglion or the cervical portion of the sympathetic trunk, such that only a portion (e.g. spatial selection) of nerve fibers and/or cell bodies within the nerve are modulated. The disclosure may additionally involve a step of selecting eye-related sympathetic nerve fibers prior to applying a signal. Methods of selective modulation of nerve fibers within a nerve are known in the art (e.g. see [references ^{16,17,18}]). Accomero et al., J. physiol. (1977), 273: 539-560.Ayres et al., J Neurophysiol. 116: 51-60(2016).Bruns et al. (2015) Neurology and Urodynamies 34: 65-71.

Where the disclosure refers to a modified eye-related sympathetic nerve, this nerve is ideally present in situ in a subject.

### Modulation of Neural Activity

According to the disclosure, applying a signal (e.g. an electrical. signal) to an eye-related sympathetic nerve results in neural activity in at least part of the nerve being modulated. Modulation of neural activity, as used herein, is taken to mean that the signaling activity of the nerve is altered from the baseline neural activity—that is, the signaling activity of the nerve in the subject prior to any intervention. Such modulation may stimulate or otherwise change the neural activity compared to baseline activity. As used herein, “neural activity” of a nerve means the signaling activity of the nerve, for example the amplitude, frequency andlor pattern of action potentials in the nerve. The term “pattern”, as used herein in the context of action potentials in the nerve, is intended to include one or more of: local field potential(s), compound action potential(s), aggregate action potential(s), and also magnitudes, frequencies, areas under the curve and other patterns of action potentials in the nerve or sub-groups (e.g. fascicules) of neurons therein.

One advantage of the disclosure is that modulation of neural activity is reversible. Hence, the modulation of neural activity is not permanent. For example, upon cessation of the application of a signal, neural activity in the nerve returns substantially towards baseline neural activity within 1-60 seconds, or within 1-60 minutes, or within 1-24 hours (e.g. within 1-12 hours, 1-6 hours, 1-4 hours. 1-2 hours), or within 1-7 days (e.g. 1-4 days, 1-2 days). In some instances of reversible modulation, the neural activity returns substantially fully to baseline neural activity. That is, the neural activity following cessation of the application of a signal is substantially the same as the neural activity prior to a signal being applied. Hence, the nerve or the portion of the nerve has regained its normal physiological capacity to propagate action potentials.

In other embodiments, modulation of the neural activity may be substantially persistent. As used herein, “persistent” is taken to mean that the modulated neural activity has a prolonged effect. For example, upon cessation of the application of a signal, neural activity in the nerve remains substantially the same as when the signal was being applied—i.e. the neural activity during and following signal application is substantially the same. Reversible modulation is preferred.

According to the disclosure, stimulation refers to neural activity in at least part of an eye-related sympathetic nerve being increased compared to baseline neural activity in that part of the nerve—that is, the signaling activity of the nerve in the subject prior to any intervention. This increase in activity can be across the whole nerve, in which case neural activity is increased across the whole nerve.

Stimulation typically involves increasing neural activity e.g. generating action potentials beyond the point of the stimulation in at least a part of the eye-related sympathetic nerve. At any point along the axon, a functioning nerve will have a distribution of potassium and sodium ions across the nerve membrane. The distribution at one point along the axon determines the electrical membrane potential of the axon at that point, which in turn influences the distribution of potassium and sodium ions at an adjacent point, which in turn determines the electrical membrane potential of the axon at that point, and so on. This is a nerve operating in its normal state, wherein action potentials propagate from point to adjacent point along the awn, and which can be observed using conventional experimentation.

One way of characterizing a stimulation of neural activity is a distribution of potassium and sodium ions at one or more points in the axon, which is created not by virtue of the electrical membrane potential at adjacent a point or points of the nerve as a result of a propagating action potential, but by virtue of the application of a temporary external electrical field. The temporary external electrical field artificially modifies the distribution of potassium and sodium ions within a point in the nerve, causing depolarization of the nerve membrane that would not otherwise occur. The depolarization of the nerve membrane caused by the temporary external electrical field generates tie novo action potential across that point. This is a nerve operating in a disrupted state, which can be observed by a distribution of potassium and sodium ions at a point in the axon (the point which has been stimulated) that has an electrical membrane potential that is not influenced or determined by a the electrical membrane potential of an adjacent paint.

Stimulation of neural activity is thus understood to be increasing neural activity from continuing past the point of signal application. Thus, the nerve at the point of signal application is modified in that the nerve membrane is reversibly depolarized by an electric field, such that a de novo action potential is generated and propagates through the modified nerve. Hence, the nerve at the point of signal application is modified in that a de novo action potential is generated.

When an electrical signal is used with the disclosure, the stimulation is based on the influence of electrical currents (e.g. charged particles, which may be one or more electrons in an electrode attached to the nerve, or one or more ions outside the nerve or within the nerve, for instance) on the distribution of ions across the nerve membrane.

Stimulation of the neural activity may be partial stimulation. Partial stimulation may be such that the total signaling activity of the whole nerve is partially increased, or that the total signaling activity of a subset of nerve fibers of the nerve is fully increased (i.e. there is no neural activity in that subset of fibers of the nerve), or that the total signaling of a subset of nerve fibers of the nerve is partially increased compared to baseline neural activity in that subset of fibers of the nerve. For example, an increase in neural activity of 5%, 10%. 15%, 20%, 25%, 30%, 35%, 40%, 45%, 50%, 60%, 70%, 80%, 90% or 95%, or an increase of neural activity in a subset of nerve fibers of the nerve. The neural activity may be measured by methods known in the art, for example, by the number of action potentials which propagate through the axon and/or the amplitude of the local field potential reflecting the summed activity of the action potentials.

The disclosure may selectively stimulate nerve fibers of various sizes within a nerve. Larger nerve fibers tend to have a lower threshold tier stimulation that smaller nerve fibers. Thus, for example, increasing signal amplitude (e.g. increasing amplitude of an electric signal) may generate stimulation of the smaller fibers as well as larger fibers. For example, asymmetrical (triangular instead of square pulse) waveforms may be used stimulate (7-fibers (unmyelinated).

Modulation of neural activity may be an alteration in the pattern of action potentials. It will be appreciated that the pattern of action potentials can be modulated without necessarily changing the overall frequency or amplitude. For example, modulation of neural activity may be such that the pattern of action potentials is altered to more closely resemble a healthy state rather than a disease state.

Modulation of neural activity may comprise altering the neural activity in various other ways, for example increasing or decreasing a particular part of the neural activity and/or stimulating new elements of activity, for example: in particular intervals of time, in particular frequency bands, according to particular patterns and so forth.

Modulation of neural activity may be (at least partially) corrective. As used herein, “corrective” is taken to mean that the modulated neural activity alters the neural activity towards the pattern of neural activity in a healthy subject, and this is called axonal modulation therapy. That is, upon cessation of signal application, neural activity in the nerve more closely resembles (ideally, substantially fully resembles) the pattern of action potentials in the nerve observed in a healthy subject than prior to signal application. Such corrective modulation can be any modulation as defined herein. For example, application of a signal may result in an increase on neural activity, and upon cessation of signal. application the pattern of action potentials in the nerve resembles the pattern of action potentials observed in a healthy subject. By way of further example, application of the signal may result in neural activity resembling the pattern of action potentials observed in a healthy subject and, upon cessation of the signal, the pattern of action potentials in the nerve remains the pattern of action potentials observed in a healthy subject.

### Eye Disorders

The disclosure is useful in treating an eye disorder. For example, the disclosure is useful in slowing, stopping or reversing progression of an eye disorder, such as an ocular neovascular disease.

The disclosure is particularly useful for treating eye disorders that are associated with ocular neovascularization, such as retinal neovascularization. For example, the disclosure is useful tier an eye disorder that is caused by or associated with the growth of blood vessels and/or blood vessel leakage in the eye. The disclosure may also be useful for treating eye disorders that have an imbalance of angiogenic growth factors compared to the physiological homeostatic state.

The disclosure may also be useful for treating an ocular neovascular disease caused by injury to the eye, e.g. by applying a signal (e.g. an electrical signal) to modulate (e.g. stimulate) the neural activity of an eye-related sympathetic nerve. For example, the eye injury may be a retinal injury, a corneal injury or conjunctival injury. The eye injury may be caused. by trauma, e.g. surgical injuries, chemical burn, corneal transplant, infectious or inflammatory diseases.

The disclosure is particularly useful for treating diabetic retinopathy (DR), e.g. by applying a signal (e.g. an electrical signal) to modulate (e.g. stimulate) the neural activity of an eye-related sympathetic nerve. DR is defined as the progressive dysfunction of the retinal vasculature caused by chronic hyperglycemia. Symptoms of DR include microaneurysms, retinal hemorrhages, retinal lipid exudates, cotton-wool spots, capillary nonperfusion, macular edema, neovascularization, increase in INF-α levels and increased retinal capillary basement membrane thickness. Associated symptoms include vitreous hemorrhage, retinal detachment, neovascular glaucoma, premature cataract and cranial nerve palsies.

DR may progress through four stages: mild nonproliferative retinopathy, moderate nonproliferative retinopathy, severe nonproliferative retinopathy and proliferative diabetic retinopathy [reference ¹⁹]. Mild nonproliferative retinopathy may be characterized by the presence of at least one microaneurysm. Moderate nonproliferative retinopathy may be characterized by multiple microaneurysms, dot-and-blot hemorrhages, venous beading, and/or cotton wool spots. A diagnosis for severe nonproliferative retinopathy is made gate patient has any of the following: diffused intraretinal hemorrhages and microaneurysms in 4 quadrants, venous beading in ≥2 quadrants, or intraretinal microvaseular abnormalities in ≥1 quadrant. Proliferative diabetic retinopathy may be characterized by retinal neovascularization, fribrovascular proliferation in the retina and vitreous fluid, vitreous hemorrhages, retinal detachment, neovascular glaucoma, severe vision loss and blindness. The disclosure may be useful in slowing, stopping or reversing the progression of DR, and/or any of the symptoms of DR.

Diabetic macular edema (DME) is the most prevalent cause of moderate vision loss in subjects with diabetes and is a common complication of DR, a disease affecting the blood vessels of the retina. Clinically significant DME occurs when fluid leaks into the center of the macula, the light-sensitive part of the retina responsible for sharp, direct vision. Fluid in the macula can cause severe vision loss or blindness. The disclosure may also be useful for treating DME, e.g. by applying a signal (e.g. an electrical signal) to modulate (e.g. stimulate) the neural activity of an eye-related sympathetic nerve.

The disclosure may also be useful for treating central retinal vein occlusion (CRVO), e.g. by applying a signal (e.g. an electrical signal) to modulate (e.g. stimulate) the neural activity of an eye-related sympathetic nerve. CRVO is caused by obstruction of the central retinal vein that leads to a back-up of blood and fluid in the retina. The retina can also become ischemic, resulting in the growth of new, inappropriate blood vessels that can cause farther vision loss and more serious complications.

A subject of the disclosure may, in addition to having an implant, receive medicine for their eye condition. For instance, a subject having an implant according to the disclosure may receive an anti-VEGF agent, e.g. an anti-VEGF antibody such as ranibizumab (which https://nei.nib.gov/health/diabetic/retinopatby. will usually continue medication which was occurring before receiving the implant). Thus the disclosure provides the use of these medicines in combination with a device or system of the disclosure.

A subject suitable for the disclosure may be any age, but will usually be at least 10, 20, 30, 40, 50, 55, 60, 65, 70, 75. 80 or 85 years of age.

### Physiological Parameters

Treatment of an eye disorder can be assessed in various ways, but typically involves determining an improvement in one or more physiological parameters of the subject. As used herein, an “improvement in a determined physiological parameter” is taken to mean that, for any given physiological parameter, an improvement is a change in the value of that parameter in the subject towards the normal value or normal range for that value—i.e. towards the expected value in a healthy subject.

As used herein, worsening of a determined physiological parameter is taken to mean that, for any given physiological parameter, worsening is a change in the value of that parameter in the subject away from the normal value or normal range for that value—i.e. away from the expected value in a healthy subject.

Useful physiological parameters of the disclosure may be one or more of the group consisting of: the level of an angiogenic growth factor in the eye, neovascularization (e.g. retinal neovascularization), ocular blood flow, blood pressure, blood oxygenation, the extent of vision impairment, the level of an immune response modulator (e. g. a cytokine) in the eye, the extent of blood vessel leakage in the eye, the extent of macular edema, the presence of retinal exudates, the presence of capillary microaneurysms, the presence of hemorrhages, the extent of retinal cell death, the extent of capillary basement membrane thickening, the level of an oxidative stress marker, and the level of a peroxynitrite marker.

For example, a subject having an eye disorder associated with retinal ocular neovascularization, such as DR, an improvement in a physiological parameter may (depending on which abnormal values a subject is exhibiting) be one or more of the group consisting of a decrease in the level of a pro-inflammatory cytokine TNF-α) in the eye, a decrease in retinal neovascularization, a decrease in retinal exudates, a decrease in capillary microaneurysms, a decrease in hemorrhages, a decrease in macular edema, a decrease in retinal cell death, a decrease in capillary basement membrane thickening, a decrease in the level of an oxidative stress marker, a decrease in the level of a peroxynitrite marker, an increase in blood oxygenation, and an improvement in vision. The disclosure might not lead to a change in all of these physiological parameters.

Suitable methods for determining the value for one or more physiological parameter will be appreciated by the skilled person. By way of example, central vision may be assessed by the Amsler Grid test. Retinal imaging is a typical way for identifying changes in the retina and macula. Commonly used retinal imaging techniques are color fundus photography, fluorescein angiography (FA), indocyanine green angiography (ICGA), optical coherence tomography (OCT), and fundus autofluorescence (FAF). For example, retinal imaging techniques can identify whether the macula is thickened or abnormal, and whether any fluid has leaked into the retina. Typically, diagnosis of DR is by funduscopy. Color fundus photography helps grade the level of retinopathy. Fluorescein angiography is used to determine the extent of retinopathy, to develop a treatment plan, and to monitor the results of treatment. Optical coherence tomography is also useful to assess severity of macular edema and treatment response.

The disclosure preferably increases the levels of anti-inflammatory cytokines in the eye, and/or decreases the levels of pro-inflammatory cytokines in the eye. Ways to measure the levels of these cytokines are known in the art. For example, the protein levels of these cytokines may be measured in a sample from the subject, e.g. in the aqueous humor, with ELISA.

Pro-inflammatory cytokines are known in the art. Examples of these include tumor necrosis tactor (TNF; also known as TNF-α or cachectin), interleukin (IL)-1α, IL-β, IL-2, IL-5, IL-6, 1L-8, 1L-15, IL-18, interferon γ (IFN-γ),platelet-activating factor (PAF), thromboxane, soluble adhesion molecules, vasoactive neuropeptides, phospholipase AZ plasminogen activator inhibitor (PAI-1), free radical generation; neopterin, CD14, prostacyclin, neutrophil elastase, protein kinase, monocyte chemotactic proteins 1 and 2 (MCP-1, MCP-2), macrophage migration inhibitory factor (MW), high mobility group box protein I (HMGB-1), and other known factors. Anti-inflammatory cytokines are also known in the art. Examples of these include IL-4, IL-10, IL-17, IL-13, IL-1α, and TNF-α receptor. It will he recognized that some of pro-inflammatory eytokines may act as anti-inflammatory cytokines in certain circumstances, and vice-versa. Such cytokines are typically referred to as pleiotropic cytokines.

The disclosure preferably reduces the level of TNF-α in the retina. For example, applying a signal (e,g an electrical signal) to stimulate an eye-related sympathetic nerve (e.g. the ICN) may cause reduction in the level of TNF-α.

The disclosure preferably decreases the levels of pro-angiogenic growth factors, such as vascular endothelial growth factor (VEGF), e.g. VEGF-A, andlor increases the levels of anti-angiogenic growth factors, such as pigment epithelial-derived factor (PEDF). PEDF is anti-angiogenic at low doses, but pro-angiogenic at high doses [reference ²⁰]. For example, applying a signal (e.g. an electrical signal) to modulate (e.g. stimulate) an eye-related sympathetic nerve (e.g, the ICN) may cause these changes.

Oxidative stress markers and peroxynitrite markers, and methods of measuring the levels of these markers, are well known in the art (e.g. see references ^{21, 22}).

In certain embodiments of the disclosure, treatment of the condition is indicated by an improvement in the profile of neural activity in the eye-related sympathetic nerve. That is, treatment of the condition is indicated by the neural activity in the eye-related sympathetic nerve approaching the neural activity in a healthy subject.

As used herein, a physiological parameter is not affected by modulation of the neural activity of the eye-related sympathetic nerve if the parameter does not change (in response to the eye-related sympathetic nerve activity modulation) from the normal value or normal range for that value of that parameter exhibited by the subject or subject when no intervention has been performed i.e. it does not depart from the baseline value for that parameter.

Preferably, modulation of the neural activity of the eye-related sympathetic nerve has minimal impact on pupil diameter. More preferably, modulation of the neural activity of R. S. Apte: et at., Investigative Ophthalmology & Visual Science, vol. 45, pp. 4491-4497, 2004Blasiak et al., BioMed Research International (2014) 768026Chiou, (2001) J. Ocul. Phamacol. Ther. (:2):189-98. the eye-related sympathetic nerve does not produce a change in pupil diameter. Changes in pupil diameter (e.g. the extent of pupil constriction) may thus be a useful indicator for optimization of the parameters of the system or device of the disclosure. If pupil diameter is affected, the methods of the disclosure could be applied while the subject is asleep.

The skilled person will appreciate that the baseline for any neural activity or physiological parameter in an subject need not be a fixed or specific value, but rather can fluctuate within a normal range or may be an average value with associated error and confidence intervals. Suitable methods for determining baseline values are well known to the skilled person.

As used herein, a physiological parameter is determined in a subject when the value for that parameter exhibited by the subject at the time of detection is determined. A detector (e.g. a physiological sensor subsystem, a physiological data processing module, a physiological sensor, etc.) is any element able to make such a determination.

Thus, in certain embodiments, the disclosure further comprises a step of determining one or more physiological parameters of the subject, wherein the signal is applied only when the determined physiological parameter meets or exceeds a predefined threshold value, in such embodiments wherein more than one physiological parameter of the subject is determined, the signal may be applied when any one of the determined physiological parameters meets or exceeds its threshold value, alternatively only when all of the determined physiological parameters meet or exceed their threshold values. In certain embodiments wherein the signal is applied by a device or system of the disclosure, the device or system further comprises at least one detector configured to determine the one or more physiological parameters of the subject.

In certain embodiments, the physiological parameter is an action potential or pattern of action potentials in a nerve of the subject, wherein the action potential or pattern of action potentials is associated with the condition that is to be treated. For example, the nerve is the eye-related sympathetic nerve. In this embodiment, the pattern of action potentials determined by the at least one detector may be associated with an eye disorder.

It will be appreciated that any two physiological parameters may be determined in parallel embodiments, the controller is coupled detect the pattern of action potentials tolerance in the subject.

A “predefined threshold value” for a physiological parameter is the minimum (or maximum) value for that parameter that must be exhibited by a subject or subject before the specified intervention is applied. For any given parameter, the threshold value may be defined as a value indicative of a pathological state or a disease state (e.g. the blood oxygenation level in the eye is greater than a threshold level, or greater than the blood oxygenation level in the eye of a healthy subject). The threshold value may be defined as a value indicative of the onset of a pathological state or a disease state. Thus, depending on the predefined threshold value, the disclosure can be used as a treatment. Alternatively, the threshold value may be defined as a value indicative of a physiological state of the subject (that the subject is, for example, asleep, post-prandial, or exercising). Appropriate values for any given physiological parameter would be simply determined by the skilled person (for example, with reference to medical standards of practice).

Such a threshold value for a given physiological parameter is exceeded if the value exhibited by the subject is beyond the threshold value that is, the exhibited value is a greater departure from the normal or healthy value for that physiological parameter than the predefined threshold value.

### An Implantable Device or System for Implementing the Disclosure

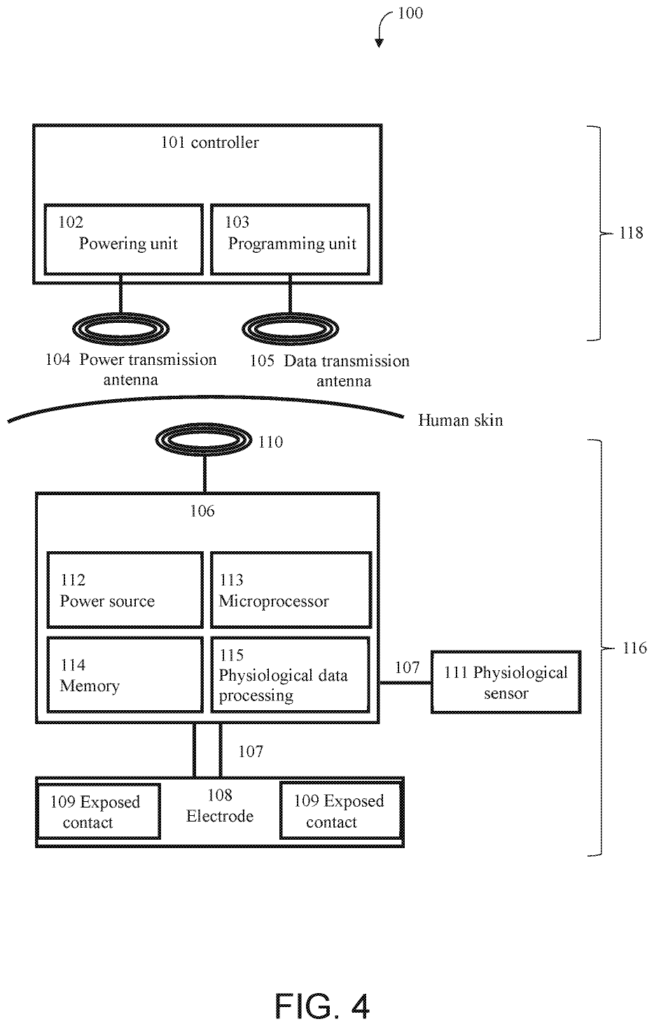

An implantable system according to the disclosure comprises an implantable device (e.g. implantable device 106 of FIG. 4). The implantable device comprises at least one neural interfacing element such as a transducer, preferably an electrode (e.g. electrode 108), suitable for placement on, in, or around an eye-related sympathetic nerve. The implantable system preferably also comprises a processor (e.g. microprocessor 113) coupled to the at least one neural interfacing element.

The at least one neural interfacing element may take many forms, and includes any component which, when used in an implantable device or system for implementing the disclosure, is capable of applying a stimulus or other signal that modulates electrical activity, e.g., action potentials, in a nerve.

The various components of the implantable system are preferably part of a single physical device, either sharing a common housing or being a physically separated collection of interconnected components connected by electrical leads (e.g. leads 107). As an alternative, however, the disclosure may use a system in which the components are physically separate, and communicate wirelessly. Thus, for instance, the at least one neural interfacing element (e.g. electrode 108) and the implantable device (e.g. implantable device 106) can be part of a unitary device, or together may form an implantable system (e.g. implantable system 116). In both cases, further components may also be present to form a larger device or system (c .g. system 100).

### Suitable Farms of a Modulating Signal

The disclosure uses a signal applied via one or more neural interfacing elements (e.g. electrode 108) placed in signaling contact with an eye-related sympathetic nerve (e.g. the ICN).

Signals applied according to the disclosure are ideally non-destructive. As used herein, a “non-destructive signal” is a signal that, when applied, does not irreversibly damage the underlying neural signal conduction ability of the nerve. That is, application of a non-destructive signal maintains the ability of the nerve (e.g. an eye-related sympathetic nerve) or fibers thereof, or other nerve tissue to which the signal is applied, to conduct action potentials when application of the signal ceases, even if that conduction is in practice artificially stimulated as a result of application of the non-destructive signal.

The signal will usually be an electrical signal, which may be, for example, a voltage or current waveform. The at least one neural interfacing element (e.g. electrode. 108) of the implantable system (e.g. implantable system 116) is configured to apply the electrical signals to a nerve, or a part thereof However, electrical signals are just one way of implementing the disclosure, as is further discussed below.

An electrical signal can take various forms, for example, a voltage or current. In certain such embodiments the signal applied comprises a direct current (DC), such as a charge-balanced DC, or a charged-balance alternating current (AC) waveform, or both a DC and an AC waveform, A combination of charge balanced DC and AC is particularly useful, with the DC being applied for a short initial period after which only AC is used [reference ²³]. As used. herein, “charge-balanced” in relation to a DC current is taken to mean that the positive or negative charge introduced into any system (e.g. a nerve) as a result of a DC current being applied is balanced by the introduction of the opposite charge in order to achieve overall (net) neutrality. In other words, a charge-balance DC current includes a Cathodic pulse and an anodic pulse.

In certain embodiments, the DC waveform or AC waveform may be a square, sinusoidal, triangular, trapezoidal, quasitrapezodial or complex waveform. The DC waveform may alternatively be a constant amplitude waveform. In certain embodiments the electrical signal is an AC sinusoidal waveform. In other embodiments, waveform comprise one or more pulse trains, each comprising a plurality of charge-balanced biphasic pulses.

The signal may be applied in bursts. The range of burst durations may be from seconds to hours; applied continuously in a duty cycled manner from 0.01% to 100%, with a predetermined time interval between bursts. The electric signal May be applied as step change or as a ramp change. in current or intensity. Particular signal parameters for modulating (e.g. stimulating) an eye-related sympathetic nerve are further described below.

Modulation of the neural activity of the eye-related sympathetic nerve can be achieved using electrical signals which serve to replicate the normal neural activity of the nerve.

With reference again to FIG. 4, the implantable system 116 comprises an implantable device 106 which may comprise a signal generator 117 (not shown); for example, a pulse generator. When the implantable device comprises a pulse generator, the implantable device 106 may be referred to as an implantable pulse generator. The signal generator 117 may also be a voltage or current source. The signal generator 117 may be pre-programmed to deliver one or more pre-defined waveforms with signal parameters falling within the range given below. Alternatively, the signal generator 117 may be controllable to adjust one or more of the signal parameters described further below. Control may be open loop, wherein the operator of the implantable device 106 may configure the signal generator Franke et al. J. Neural Eng. 2014; 11(5):056012. using an external controller (e.g. controller 101), or control may be closed loop, wherein. signal generator modifies the signal parameters in response to one or more physiological parameters of the subject, as is further described below.

### Signal Parameters Jar Modulating Neural Activity

In all of the above examples, the signal generator 117 may be. configured to deliver an electrical signal for modulating (e.g. stimulating) an eye-related sympathetic nerve (e.g. the ICN). In the present application, the signal generator 117 is configured to apply an electrical signal with certain signal parameters to modulate (e.g. stimulate) neural activity in an eye-related sympathetic nerve (e.g. the ICN). Signal parameters for modulating (e.g. stimulating) the eye-related sympathetic nerve, which are described herein, may include waveform, amplitude and frequency.

In certain embodiments for stimulating neural activity in an eye-related sympathetic nerve, the electrical signal has a frequency of 1 Hz to 50 Hz. Whilst frequencies of between 1 Hz and 50 Hz are possible, frequencies between 1 Hz and 30 Hz are expected to be more viable and frequencies between 1 Hz and 20 Hz more viable still. Frequencies of 1 Hz, 5 Hz and particularly 10 Hz are preferred, though any frequency within the range may be chosen.

The signal generator 117 may he configured to deliver one or more pulse. trains at intervals according to the above-mentioned frequencies. For example, a frequency of 1 to 50 Hz results in a pulse interval between 1 pulse per second and 50 pulses per second, within a given pulse train. The range of pulse widths may be from 0.01 to 2 ins (including, if applicable, both positive and negative phases of the pulse, in the case of a charge-balanced biphasic pulse). The range of pulse amplitudes may be from 0.01 to 10 mA peak-to-peak. For stimulating neural activity, advantages have noted in respect of pulses of shorter pulse widths and lower amplitudes. In particular pulse widths between 0.2 ms and 0.5 ms and pulse amplitudes between 0.35 mA and 0.60 mA are preferred, though waveforms with pulse widths between 50 μs and 1 ms and pulse amplitudes between 0.20 mA and 0.65 mA are also advantageous.

In certain embodiments for stimulating neural activity in an eye-related sympathetic nerve, the electrical signal has a current between 0.1 to 5 mA, preferably between 0.35 mA and 1 mA, preferably between 0.60 mA and 0.65 mA. It will be appreciated by the skilled person that the current amplitude of an applied electrical signal necessary to achieve the intended modulation of the neural activity will depend upon the positioning of the electrode and the associated electrophysiological characteristics (e.g. impedance). It is within the ability of the skilled person to determine the appropriate current amplitude for achieving the intended modulation of the neural activity in a given subject.

Electrodes

As mentioned above, the implantable system comprises at least one neural interfacing element, the neural interfacing element is preferably an electrode 108. The neural interface is configured to at least partially and preferably fully circumvent the eye-related sympathetic nerve. The geometry of the neural interface is defined in part by the anatomy of the eye-related sympathetic nerve. In particular, the geometry may be limited by the length of the eye-related sympathetic nerve and/or by the diameter of the eye-related sympathetic nerve. For example, the dimensions of the ganglia useful with the disclosure are shown in Table 1.

TABLE 1
Measurements of the superior cervical ganglion, single
middle cervical ganglion and the inferior cervical/cervicothoracic
ganglion [reference ^{24]}.

| | | Mean | Min. | Max. |
|---|---|---|---|---|
| | | (mm) | (mm) | (mm) |
| Superior cervical ganglion | Length | 33.0 ± 6.2 | 13.1 | 45.7 |
| | Width | 8.1 ± 5.4 | 3.8 | 17.6 |
| Single middle cervical | Length | 8.9 ± 45.4 | 3.0 | 21.6 |
| ganglion | Width | 5.1 ± 2.1 | 2.9 | 9.6 |
| Inferior cervical/ | Length | 11.3 ± 4.6 | 5.1 | 23 |
| cervicothoracic ganglion | Width | 8.2 ± 3.0 | 3.5 | 15.6 |

In some embodiments (for example, FIG. 4), electrode 108 may be coupled to implantable device 106 of implantable system 116 via electrical leads 107. Alternatively, implantable device 106 may be directly integrated with the electrode 108 without leads. In any case, implantable device 106 may comprise DC current blocking output circuits, optionally based on capacitors and/or inductors, on all output channels (e.g. outputs to the electrode 108, or physiological sensor 111). Electrode 108 may be shaped as one of: a rectangle, an oval, an ellipsoid, a rod, a straight wire, a curved wire, a helically wound wire, a barb, a hook, or a cuff. In addition to electrode 108 which, in use, is located on, in, or near an eye-related sympathetic nerve ((e.g. the ICN), there may also be a larger indifferent electrode placed 119 (not shown) in the adjacent tissue.

Preferably, electrode 108 may contain at least two electrically conductive exposed contacts 109 configured, in use, to be placed on, in, or near an eye-related sympathetic nerve to innervate the eye. Exposed contacts 109 may be positioned, in use, transversely along the axis of an eye-related sympathetic nerve. In this configuration, the distance between each of the at least two exposed contacts may be between about a 0.5 mm and about 5 mm, optionally between about 1 mm and 3 mm, optionally between about 1 mm and 2 mm. Each of the at least two exposed contacts 109 may have a surface area in contact with an eye-related sympathetic nerve which is equal to that of the other. The surface area may range between Saylam et al. clinical Anatomy, 22:324-330. about 0.1 mm² and about 100 mm², optionally between about 1 mm² to 50 mm², optionally between about 1 mm² to 20 mm², optionally about 5 mm² to 10 mm².

A particularly preferred form of electrode 108 for use in the present disclosure is an electrode array. Electrode arrays are capable of stimulating the nerve in a spatially selective manner, as is known (see, e.g. [references 16, 17, 18]).

The electrode arrays may be of the penetrating or non-penetrating type. A suitable electrode array may be an ICS-96 MultiPort planar array from Blackrock Microsystems, One possible configuration has 90 channels: 4×10 and 5×10 split planar arrays, with approximately 2000 mm² surface area, 1 mm shaft length, and 0.4 mm interelectrode spacing.

Exposed contacts 109 may be insulated by a non-conductive biocompatible material, which may be spaced transversely along the eye-related sympathetic nerve in use.

### Other Suitable Prins of Neural Interfacing Element and Signal

The signal may comprise an electromagnetic signal, such as an optical Optical signals can conveniently be applied using a laser and/or a light emitting diode configured to apply the optical signal. Optogenetics is a technique in which genetically-modified cells express photosensitive features which can then be activated with light to modulate cell function. Many different optogenetic tools have been developed, for stimulating neural firing. A list of optogenetic tools to suppress neural activity is compiled in [reference ²⁵]. Thus light can be used with genetic modification of target cells to achieve stimulation of neural activity. Kramer et al., Optogenetic pharmacology for control of native neuronal signaling proteins, 2013; 16(7): 816-23.

The signal may use thermal energy, and the temperature of a nerve can be modified to stimulate the propagation of neural activity. Heating the nerve can be used to modulate neural activity. In certain such embodiments, the neural interface is a small implantable or wearable transducer or device tier radiant electromagnetic heating using visible, infrared, or Microwave radiation, for example using a laser diode or a light emitting diode. In certain such embodiments, the radiant signal has an energy density less than 500 mW/cm². Further, in certain embodiments, the radiant signal is modulated with a modulation frequency of less than 5 Hz, optionally 1 Hz. In certain alternative embodiments, the neural interface is a small implantable or wearable transducer or device for conductive heating, such as an electrically resistive element, which can be used to provide a fast, reversible, and spatially very localized heating effect (see for example reference [reference ²⁶]. In certain embodiments, the signal increases the temperature of the nerve by up to 5° C. Duke et al., (2012), J. Neural Eng., 9(3): 036003.

The signal may comprise a mechanical signal. In certain embodiments, the mechanical signal is a pressure signal. In certain such embodiments, the neural interface is a transducer which generates pressure of up to 250 mmHg to be applied to the nerve which stimulates neural activity.

In certain alternative embodiments, the signal is an ultrasonic signal. In certain such embodiments, the neural interface is an ultrasound transducer, and the ultrasonic signal has a frequency below 0.5 MHz, optionally 0.1-0.5 MHz, optionally 0.1 MHz. In certain embodiments, the ultrasonic signal has a density of below 10 W/cm, for example 1.5 W/cm² or 95 W/cm².

Another mechanical form of signal for modulating neural activity uses ultrasound which may conveniently be implemented using external, for example wearable, instead of implanted, ultrasound transducers.

### Microprocessor

The implantable system 116, in particular the implantable device 106, may comprise a processor, for example microprocessor 113. Microprocessor 113 may be responsible for triggering the beginning and/or end of the signals delivered to the nerve (e.g., an eye-related sympathetic nerve) by the at least one neural interfacing element Optionally, microprocessor 113 may also be responsible for generating and/or controlling the parameters of the signal.

Microprocessor 113 may be configured to operate in an open-loop fashion, wherein a pre-defined signal (e.g. as described above) is delivered to the nerve at a given periodicity (or continuously) and for a given duration (or indefinitely) with or without an external trigger, and without any control or feedback mechanism. Alternatively, microprocessor 113 may be configured to operate in a closed-loop fashion, wherein a signal is applied based on a control or feedback mechanism. As described elsewhere herein, the external trigger may be an external controller 101 operable by the operator to initiate delivery of a signal.

Microprocessor 113 of the implantable system 116, in particular of the implantable device 106, may be constructed so as to generate, in use, a preconfigured and/or operator-selectable signal that is independent of any input. Preferably, however, microprocessor 113 is responsive to an external signal, more preferably information (e.g. data) pertaining to one or more physiological parameters of the subject.

Microprocessor 113 may be triggered upon receipt of a signal generated by an operator, such as a physician or the subject in which the device 116 is implanted. To that end, the implantable system 116 may be part of a system which additionally comprises an external system 118 comprising a controller 101. An example of such a system is described below with reference to FIG. 4.

External system 118. of system 100 is external the implantable system 116 and external to the subject, and comprises controller 101. Controller 101 may be used for controlling and/or externally powering implantable system 116. To this end, controller 101 may comprise a powering unit 102 and/or a programming unit 103. The external system 118 may further comprise a power transmission antenna 104 and a data transmission antenna 105, as further described below.

The controller 101 and/or microprocessor 113 may be configured to apply any one or more of the above signals to the nerve intermittently or continuously. Intermittent application of a signal involves applying the signal in an (on-off) pattern, where n>1. For instance, the signal can be applied continuously for at least 5 days, optionally at least 7 days, before ceasing for a period (e.g. 1 day, 2 days, 3 days, 1 week, 2 weeks, 1 month), before being again applied continuously for at least 5 days, etc. Thus the signal is applied for a first time period, then stopped for a second time period, then reapplied for a third time period, then stopped for a fourth time period. etc. In such an embodiment, the first, second, third and fourth periods run sequentially and consecutively. The duration of the first, second, third and fourth time periods is independently selected. That is, the duration of each time period may be the same or different to any of the other time periods. In certain such embodiments, the duration of each of the first, second, third and fourth time periods may be any time from 1 second (s) to 10 days (d). 2s to 7d, 3s to 4d, 5s to 24 hours (24 h), 30s to 12 h, 1 min to 12 h, 5 min to 8 h, 5 min to 6 h, 10 min to 6 h, 10 min to 4h, 30 min to 4 h, 1 h to 4 h. In certain embodiments, the duration of each. of the first, second, third and fourth time periods is 5s, 10s, 30s, 60s, 2 min, 5 min, 10 min, 20 min, 30 min, 40 min, 50 min, 60 min, 90 min, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, 2d, 3d, 4d, 5d, 6d, 7d.

In certain embodiments, the signal is applied by controller 101 and/or microprocessor for a specific amount of time per day. in certain such embodiments, the signal is applied for 10 min, 20 min, 30 min, 40 min, 50 min, 60 min, 90 min, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h per day. In certain such embodiments, the signal is applied continuously for the specified amount of time. in certain alternative such embodiments, the signal may be applied discontinuously across the day, provided the total time of application amounts to the specified time.

Continuous application may continue indefinitely, e.g. permanently. Alternatively, the continuous application may be for a minimum period, for example the signal may be continuously applied for at least 5 days, or at least 7 days.

Whether the signal .applied to the nerve is controlled by controller 101, or whether the signal is continuously applied directly by microprocessor 113, although the signal might be a series of pulses, the gaps between those pulses do not mean the signal is not continuously applied.

In certain embodiments, the signal is applied only when the subject is in a specific state e.g., only when the subject is awake, only when the subject is asleep, prior to and/or after the ingestion of food, prior to and/or after the subject undertakes exercise, etc.

The various embodiments for timing for modulation of neural activity in the nerve can all be achieved using controller 101 in a device or system of the disclosure.

### Other Components of the System Including the Implantable Device

In addition to the aforementioned electrode 108 and microprocessor 113, the implantable system 116 may comprise one or more of the following components: implantable transceiver 11.0; physiological sensor 111; power source 112; memory 114; and physiological data processing module 115. Additionally or alternatively, the physiological sensor 111; memory 114; and physiological data processing module 115 may be part of a sub-system external to the implantable system. Optionally, the external sub-system may be capable of communicating with the implantable system, for example wirelessly via the implantable transceiver 110.

In some embodiments, one or more of the following components may preferably be contained in the implantable device 106: power source 112; memory 114; and a physiological data processing module 115.

The power source 112 may comprise a current source and/or a voltage source for providing the power for the signal delivered to an eye-related sympathetic nerve by the electrode 108. The power source 112 may also provide power for the other components of the implantable device 106 and/or implantable system 116, such as the microprocessor 113, memory 114, and implantable transceiver 110. The power source 112 may comprise a battery, the battery may be rechargeable.

It will be appreciated that the availability of power is limited in implantable devices, and the disclosure has been devised with this constraint in mind. The implantable device 106 and/or implantable system 116 may be powered by inductive powering or a rechargeable power source.

Memory 114 may store power data and data pertaining to the one or more physiological parameters from internal system 116. For instance, memory 114 may store data pertaining to one or more signals indicative of the one or more physiological parameters detected by physiological sensor 111, and/or the one or more corresponding physiological parameters determined via physiological data processing module 115. In addition or alternatively, memory 114 may store power data and data pertaining to the one or more physiological parameters from external system 118 via the implantable transceiver 110. To this end, the implantable transceiver 110 May form part of a communication subsystem of the system 100, as is further discussed below.

Physiological data processing module 115 is configured to process one or more signals indicative of one or more physiological parameters detected by the physiological sensor 111, to determine one or more corresponding physiological parameters. Physiological data processing. module 115 may be configured for reducing the size of the data pertaining to the one or more physiological parameters for storing in memory 114 and/or for transmitting to the external system via implantable transceiver 110. Implantable transceiver 110 may comprise an one or more antenna(e). The implantable transceiver 100 may use any suitable signaling process such as RF, wireless, infrared and so on, for transmitting signals outside of the body, for instance to system 100 of which the implantable system 116 is one part.

Alternatively or additionally, physiological data processing module 115 may be configured to process the signals indicative of the one or more physiological parameters and/or process the determined one or more physiological parameters to determine the evolution of the eye-related medical condition in the subject. In such case, the implantable system 116, in particular the implantable device 106, will include a capability of calibrating and tuning the signal parameters based on the one or more physiological parameters of the subject and the determined evolution of the eye-related medical condition in the subject, as is further discussed below.

The physiological data processing module 115 and the at least one physiological sensor 111 may form a physiological sensor subsystem, also known herein as a detector, either as part of the implantable system 116, part of the implantable device 106, or external to the implantable system.

Physiological sensor 111 comprises one or more sensors, each configured to detect a signal indicative of one of the one or more physiological parameters described above. For example, the physiological sensor 110 is configured for one or more of: detecting electrodermal activity using an electrical sensor; detecting electroretinographic activity using an electrical sensor; detecting biomolecule concentration using electrical, RF or optical (visible, infrared) biochemical sensors; or a combination thereof.

The physiological parameters determined by the physiological data processing module 115 may be used to trigger the microprocessor 113 to deliver a signal of the kinds described above to an eye-related sympathetic nerve using the electrode 108. Upon receipt of the signal indicative of a physiological parameter received from physiological sensor 111, the physiological data processor 115 may determine the physiological parameter of the subject, and the evolution of the eye-related medical condition, by calculating in accordance with techniques known in the art.

The memory 114 may store physiological data pertaining to normal levels of the one or more physiological parameters. The data may be specific to the subject into which. the implantable system 116 is implanted, and gleaned from various tests known in the art. Upon receipt of the signal indicative of a physiological parameter received from physiological sensor 111, or else periodically or upon demand from physiological sensor 111, the physiological data processor 115 may compare the physiological parameter determined from the signal received from physiological sensor 111 with the data pertaining to a normal level of the physiological parameter stored in the memory 114, and determine whether the received signals are indicative of insufficient or excessive of a particular physiological parameter, and thus indicative of the evolution of the eye-related medical condition in the subject.

The implantable system 116 and/or implantable device 106 may be configured such that if and when an insufficient or excessive level of a physiological parameter is determined by physiological data processor 115, the physiological data processor 115 triggers delivery of a signal to an eye-related sympathetic nerve by the neural interface (e.g. electrode 108), in the manner described elsewhere herein. For instance, if physiological parameter indicative of worsening of any of the physiological parameters and/or of the disease is determined, the physiological data processor 115 may trigger delivery of a signal which dampens secretion of the respective biochemical, as described elsewhere herein. Particular physiological parameters relevant to the present disclosure are described above. When one or more signals indicative of one or more of these physiological parameters are received by the physiological data processor 115, a signal may be applied to an eye-related sympathetic nerve via the electrode 108.

As an alternative to, or in addition to, the ability of the implantable system 116 and/or implantable device 106 to respond to physiological parameters of the subject, the microprocessor 113 may be triggered upon receipt of a signal generated by an operator (e.g. a physician or the subject in which the system 116 is implanted). To that end, the implantable system 116 may be part of a system 100 which comprises external system 118 and controller 101, as is further described below.

### System Including Implantable Device

With reference to FIG. 4, the implantable device 106 of the disclosure may be part of a system 110 that includes a number of subsystems, for example the implantable system 116 and the external system 118. The external system 118 may be used for powering and programming the implantable system 116 and/or the implantable device 106 through human skin and underlying tissues.

The external subsystem 118 may comprise, in addition to controller 101, one or more of: a powering unit 102, for wirelessly recharging the battery of power source 112 used to power the implantable device 106; and, a programming unit 103 configured to communicate with the implantable transceiver 110. The programming unit 103 and the implantable transceiver 110 may form a communication subsystem. In sonic embodiments, powering unit 102 is housed together with programing unit 103. In other embodiments, they can be housed in separate devices.

The external subsystem 118 may also comprise one or more of: power transmission antenna 104; and data transmission antenna 105. Power transmission antenna 104 may be configured for transmitting an electromagnetic field at a low frequency (e.g., from 30 kHz to 10 MHz). Data transmission antenna 105 may be configured to transmit data for programming or reprogramming the implantable device 106, and may he used in addition to the power transmission antenna 104 for transmitting an electromagnetic field at a high frequency (e.g., from 1 MHz to 10 GHz). The temperature in the skin will not increase by more than 2 degrees Celsius above the surrounding tissue during the operation of the power transmission antenna 104. The at least one antennae of the implantable transceiver 110 may be configured to receive power from the external electromagnetic field generated by power transmission antenna 104, Which may be used to charge the rechargeable battery of power source 112.

The power transmission antenna 104, data transmission antenna 105, and the at least one antennae of implantable transceiver 110 have certain characteristics such a resonant frequency and a quality factor (Q). One implementation of the antenna(e) is a coil of wire with or without a ferrite core forming an. inductor with a defined inductance. This inductor may be coupled with a resonating capacitor and a resistive loss to form the resonant circuit, The frequency is set to match that of the electromagnetic field generated by the power transmission antenna 105, A second antenna of the at least one antennae of implantable transceiver 110 can be used in implantable system 116 for data reception and transmission from/to the external system 118. If more than one antenna is used in the implantable system 116, these antennae are rotated 30 degrees from one another to achieve a better degree of power transfer efficiency during slight misalignment with the with power transmission antenna 104.

External system 118 may comprise one or more external body-worn physiological sensors 121 (not shown) to detect signals indicative of one or more physiological parameters. The signals may he transmitted to the implantable system 116 via the at least one antennae of implantable transceiver 110. Alternatively or additionally, the signals may be transmitted to the external system 116 and then to the implantable system 116 via the at least one antennae of implantable transceiver 110. As with signals indicative of one or more physiological parameters detected by the implanted physiological sensor 111, the signals indicative of one or more physiological parameters detected by the external sensor 121 may be processed by the physiological data processing module 115 to determine the one or more physiological parameters and./or stored in memory 114 to operate the implantable system 116 in a closed-loop fashion. The physiological parameters of the subject determined via signals received from the external sensor 121 may be used in addition to alternatively to the physiological parameters determined via signals received from the implanted physiological sensor 111.

For example, in a particular embodiment a detector external to the implantable device may include an optical detector including a camera capable of imaging the eye and determining changes in physiological parameters, in particular the physiological parameters described above. As explained above, in response to the determination of one or more of these physiological parameters the detector may trigger delivery of signal to an eye-related sympathetic nerve by the electrode 108, or may modify the parameters of the signal being delivered or a signal to be delivered to an eye-related sympathetic nerve by the electrode 108 in the future.

The system 100 may include a safety protection feature that discontinues the electrical stimulation of an eye-related sympathetic nerve in the following exemplary events: abnormal operation of the implantable system 116 (e.g. overvoltage); abnormal readout from air implanted physiological sensor 111 (e.g. temperature increase of More than 2 degrees Celsius or excessively high or low electrical impedance at the electrode-tissue interface); abnormal readout from an external body-worn physiological sensor 121 (not shown); or abnormal response to stimulation detected by an operator (e.g. a physician or the subject). The safety precaution feature may be implemented via controller 101 and communicated to the implantable system 116, or internally within the implantable system 116.

The external system 118 may comprise an actuator 120 (not shown) which, upon being pressed by an operator (e.g. a physician or the subject), will deliver a signal, via controller 101 and the respective communication subsystem, to trigger the microprocessor 113 of the implantable system 116 to deliver a signal to the nerve by the electrode 108.

System 100 of the disclosure including the external system 118, but in particular implantable system 116, is preferably made from, or coated with, a biostable and biocompatible material. This means that the device or system is both protected from damage due to exposure to the body's tissues and also minimizes the risk that the device or system elicits an unfavorable reaction by the host (which could ultimately lead to rejection). The material used to make or coat the device or system should ideally resist the formation of biofilms. Suitable materials include, but are not limited to, polyp-xylylene) polymers (known as Parylenes) and polytetrafluoroethylene.

The implantable device 116 of the disclosure will generally weigh less than 50 g.

### General

The term “comprising” encompasses “including” as well as “consisting” e.g. a composition “comprising” X may consist exclusively of X or may include something additional e.g. X+Y.

The word “substantiaIly”does not exclude “completely” e.g. a composition which is “substantially free” from Y may be completely free from Y. Where necessary, the word “substantially” may be omitted from the definition of the disclosure.

The term “about” in relation to a numerical value x is optional and means, for example, x±10%.

Unless otherwise indicated each embodiment as described herein may be combined with another embodiment as described herein,

### Modes for Carrying out the Disclosure

The aim of the experimental study was to test the validity of neural modulation. In particular, the experimental study aimed to evaluate the effects of ICN denervation on pro-inflammatory cytokine levels in the retina, e.g., the effects of unilateral ICN denervation on retinal TNF-α protein levels were measured in the ipsilateral versus contralateral (control) eye. Elevated retinal TNF-α levels are observed in patients with DR; thus, this study tested. whether ICN denervation produced a phenotype resembling that of DR.

### Methods

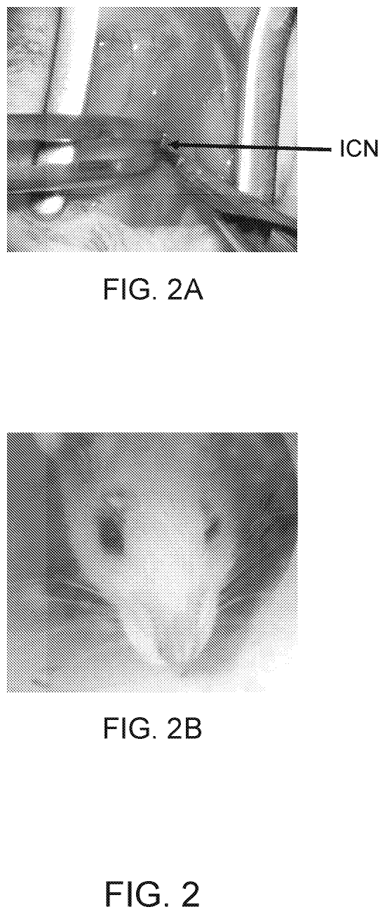

Female Sprague Dawley rats (n=5), and ˜P60, underwent unilateral ICN transection. For ICN transection, rats were anesthetized with ketamine/xylazine placed in the supine position in order to expose ventral structures of the neck. Upper limbs were extended, providing better exposition of the surgical area. A vertical incision was made in the middle of the neck. The incision began 2 cm below the intermandibular region in the presternal region, The skin was retracted, and tissue underneath was dissected by blunt dissection, including superficial cervical fascia with mandibular glands. Neck muscles were exposed (sternohyoid, omohyoid, sternomastoid, and posterior belly of the digastric muscles), and the carotid triangle was located between the muscles. Within the triangle, the carotid bifurcation was identified and separated into its structures (external and internal carotid arteries). The occipital artery and hypoglossal nerve were clearly observed. The SCG was identified below those structures, and the internal and external carotid nerves were exposed. The ICN was fully transected distal to the SCG, beneath/adjacent to the hypoglossal nerve (FIG. 2A). Following transection, the skin incision was closed with a non-absorbable suture (nylon 6-0), and antibiotic ointment was applied. To verify successful surgery, eyelid and eyeball position were evaluated over the next 3 days. Ptosis of the ipsilateral eyelid was generally observed within 4-12 hrs after surgery, followed by exophthalmos between 12-24 hrs. Permanent ptosis ensued ˜24 hrs after ICN transection (FIG. 28). Animals without apparent ptosis were euthanized.

Animals were euthanized 6 weeks after ICN transection, and eyes were enucleated. The retinas were isolated from each eye. Control and denervated retinas were pooled separately and homogenized in 250 μL of buffer (80 mM Tris-HCl, 4 mM MgCl2, 0.5 mM phenylmethylsulfonyl fluoride) containing mixed protease inhibitors (Roche, Basel, Switzerland) for protein extraction. Protein homogenates were centrifuged at 14,000 g for 10 min to remove tissue debris. Total protein concentration was determined by a Bio-Rad protein assay (Bio-Rad Laboratories, Hercules, Calif.). TNF-α protein expression in the retinas Was assessed with 100 μg total protein per sample in triplicate with a TNF-α ELISA kit (Cell Applications, San Diego, Calif.). The detection range of this assay is 15-1000 pg/mL,

### Results and Discussion

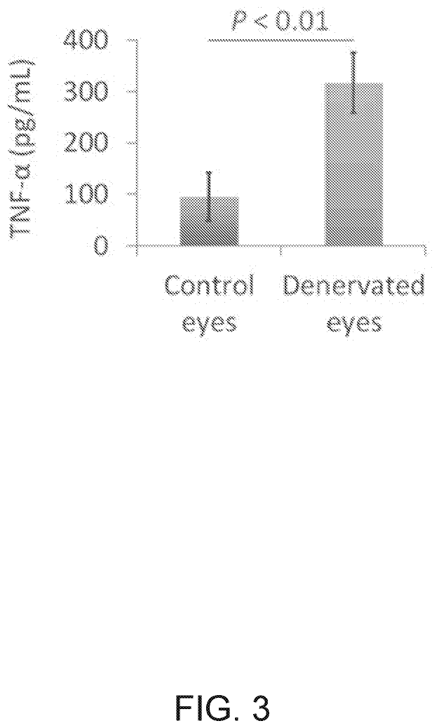

It was found that ICN transection caused elevated retinal TNF-α levels. TNF-α protein levels in denervated retinas increased by 3.3-fold relative to protein levels in contralateral retinas (see FIG. 3). This finding resembles those in [reference ²⁷] and [reference ²⁸], in which β-AR receptor knockout mice exhibited 20-30% higher retinal TNF-α protein levels than wild-type mice. Because upregulation of inflammatory cytokines such as TNF-α is implicated in the pathogenesis of DR, the results suggest that electrical stimulation of the ICN could serve as a possible treatment fir the disease. Jiang et al., 2013, PLoS One, 8(7), 0055.Panjala et al., 2011, Molecular Vision, 17, 1822-1828.

Hence, this study suggests that electrical modulation (e.g. stimulation) of the ICN activity could be an effective strategy for treating eye disorders, e.g eye disorders that are associated with retinal neovascularization, such as DR, or ocular neovascular diseases caused by injury to the eye.

1.-32. (canceled)

33. A device or system comprising at least one neural interfacing electrode placed on, in, or around an eye-related sympathetic nerve, and a voltage or current source configured to generate an electrical signal to be applied to the eye-related sympathetic nerve via the at least one neural interfacing electrode wherein the electrical signal reversibly stimulates neural activity of the eye-related sympathetic nerve to produce a change in a physiological parameter in a subject, wherein the physiological parameter is one or more of the group consisting of: a level of an angiogenic growth factor in the eye, neovascularization ocular blood flow, blood pressure, blood oxygenation, an extent of vision impairment, a level of an immune response modulator in the eye, an extent of blood vessel leakage in the eye, an extent of macular edema, a presence of retinal exudates, a presence of capillary microaneurysms, a presence of hemorrhages, an extent of retinal cell death, an extent of capillary basement membrane thickening, a level of an oxidative stress marker, and a level of a peroxynitrite marker.

34. The device or system of claim 33, wherein the eye-related sympathetic nerve is modulated at an internal carotid nerve (ICN).

35. The device or system of claim 33, wherein the eye-related sympathetic nerve is modulated unilaterally or bilaterally.

36. The device or system of claim 33, wherein the electrical signal comprises a charge-balanced DC signal and/or a charge-balanced AC signal.

37. The device or system of claim 33, wherein the change in the physiological parameter is one or more of the group consisting of: a decrease in the level of a pro-inflammatory cytokine in the eye, a decrease in retinal neovascularization, a decrease in retinal exudates, a decrease in capillary microaneurysms, a decrease in hemorrhages, a decrease in macular edema, a decrease in retinal cell death, a decrease in capillary basement membrane thickening, a decrease in the level of an oxidative stress marker, a decrease in the level of a peroxynitrite marker, an increase in blood oxygenation in the eye, and an improvement in vision.

38. The device or system of claim 33, wherein the electrical signal has a frequency between 1 Hz and 50 Hz.

39. The device or system of claim 33, comprising a detector for detecting one or more signals indicative of one or more physiological parameters; determining from the one or more signals one or more physiological parameters; determining the one or more physiological parameters indicative of worsening of the physiological parameter; and causing the signal to be applied to the eye-related sympathetic nerve via the at least one electrode.

40. The device or system of claim 39, further comprising a memory for storing data pertaining to physiological parameters in a healthy subject, wherein determining the one or more physiological parameters indicative of worsening of the physiological parameter comprises comparing the one or more physiological parameters with the data.

41. The device or system of claim 33, comprising a communication subsystem for receiving a control signal from a controller and, upon detection of said one or more control signals, cause the electrical signal to be applied to the eye-related sympathetic nerve via the at least one electrode.

42. A method of reversibly stimulating neural activity in the internal carotid nerve (ICN) comprising (i) implanting in a subject a device or system comprising at least one neural interfacing electrode placed on, in, or around the ICN, and a voltage or current source configured to generate an electrical signal to be applied to the ICN via the at least one neural interfacing electrode wherein the electrical signal reversibly stimulates neural activity of the ICN to produce a change in a physiological parameter in a subject, wherein the physiological parameter is one or more of the group consisting of: a level of an angiogenic growth factor in the eye, neovascularization, ocular blood flow, blood pressure, blood oxygenation, an extent of vision impairment, a level of an immune response modulator in the eye, an extent of blood vessel leakage in the eye, an extent of macular edema, a presence of retinal exudates, a presence of capillary microaneurysms, a presence of hemorrhages, an extent of retinal cell death, an extent of capillary basement membrane thickening, a level of an oxidative stress marker, and a level of a peroxynitrite marker.

43. The method of claim 42, wherein the method decreases retinal neovascularization.

44. The method of claim 42, wherein the method treats an eye disorder associated with ocular neovascularization.

45. The method of claim 42, wherein the change in the physiological parameter is one or more of the group consisting of: a decrease in the level of a pro-inflammatory cytokine in the eye, a decrease in retinal neovascularization, a decrease in retinal exudates, a decrease in capillary microaneurysms, a decrease in hemorrhages, a decrease in macular edema, a decrease in retinal cell death, a decrease in capillary basement membrane thickening, a decrease in the level of an oxidative stress marker, a decrease in the level of a peroxynitrite marker, an increase in blood oxygenation in the eye, and an improvement in vision.

46. The method of claim 42, wherein the electrical signal has a frequency between 1 Hz and 50 Hz.

47. The method of claim 42, wherein the method is for treating diabetic retinopathy or an ocular neovascular disease caused by injury to the eye.

48. A method of reversibly stimulating neural activity in an eye-related sympathetic nerve, comprising: (i) implanting in a subject a device or system of claim 33 and (ii) positioning the neural interfacing element in signaling contact with the eye-related sympathetic nerve.

49. The method of claim 48, wherein the method is for treating an eye disorder, such as an ocular neovascular disease.

50. The method of claim 49, wherein the method is for treating diabetic retinopathy or an ocular neovascular disease caused by injury to the eye.

51. A method for treating an eye disorder, comprising applying an electrical signal to an eye-related sympathetic nerve via at least one neural interfacing electrode, wherein the signal reversibly stimulates neural activity of the eye-related sympathetic nerve to produce a change in a physiological parameter in a subject, wherein the at least one neural interfacing electrode is suitable for placement on, in, or around the eye-related sympathetic nerve, wherein the physiological parameter is one or more of the group consisting of: the level of an angiogenic growth factor in the eye, neovascularization, ocular blood flow, blood pressure, blood oxygenation, an extent of vision impairment, a level of an immune response modulator in the eye, an extent of blood vessel leakage in the eye, an extent of macular edema, a presence of retinal exudates, a presence of capillary microaneurysms, a presence of hemorrhages, an extent of retinal cell death, an extent of capillary basement membrane thickening, a level of an oxidative stress marker, and a level of a peroxynitrite marker.

52. The method of claim 51, wherein the eye disorder is diabetic retinopathy or an ocular neovascular disease caused by injury to the eye.
